(19) United States
(12) Patent Application Publication
(10) Pub. No.: US 2013/0084259 A1
(43) Pub. Date: Apr. 4, 2013

(54) METHOD FOR PREPARING CUSTOMIZED COSMETICS

(71) Applicant: Sung Reol Lee, Jeonbuk (KR)

(72) Inventor: Sung Reol Lee, Jeonbuk (KR)

(21) Appl. No.: 13/703,141

(22) PCT Filed: Jun. 8, 2011
(86) PCT No.: PCT/KR2011/004184

§ 371 Date: Dec. 10, 2012

(30) Foreign Application Priority Data
Jun. 9, 2010 (KR) 10-2010-0054457

(51) Int. Cl. A61K 8/64; A61Q 13/00; A61Q 1/02; A61K 8/97; A61K 8/67

(52) U.S. Cl. CPC A61K 8/64; A61K 8/97; A61K 8/678; A61Q 1/02; A61Q 13/00
USPC 424/63; 512/1

(57) ABSTRACT

The present disclosure provides a method of manufacturing customized cosmetics in which a conventional cosmetics production process for producing complete products in a factory is segmented such that a base product, functional material and/or additive are produced or prepared in a factory individually, and a user may select the functional material and/or additive in accordance with usage preference and/or skin conditions of the user and mix the selected functional material and/or additives with the base product, thereby satisfying various needs of users and enabling each individual to prepare cosmetics tailored to him/her.

### CROSS-REFERENCE TO RELATED APPLICATION

This application claims the benefit of Korean Patent Application No.10-2010-0054457, filed on Jun. 9, 2010 in the Korean Intellectual Property Office, the disclosure of which is incorporated herein by reference.

### FIELD OF INVENTION

The present disclosure relates to a method of manufacturing customized cosmetics, and more particularly, to a method of manufacturing customized cosmetics in which a conventional cosmetics production process for producing complete products in a factory is segmented such that a base product, functional material and/or additive are produced or prepared in a factory individually, and a user may select the functional material and/or additive in accordance with usage preference and/or skin conditions of the user and mix the selected functional material and/or additives with the base product, thereby satisfying various needs of users and enabling each individual to prepare cosmetics tailored to him/her.

### BACKGROUND

Current commercially available cosmetics are manufactured by a batch process with a large quantity in a factory to be sold as end-products or complete products with a variety of functions to be directly used by a consumer. This batch process to manufacture the complete cosmetics has been common because pursuant to national regulations relating to manufacturing the cosmetics, only manufacturers having prescribed manufacturing and quality management facilities and thus being accredited by the government may make the commercially available cosmetics.

Such a conventional method of manufacturing the cosmetics is advantageous in that each process may be easily monitored to prevent the resultant product from being harmful to the consumer, thus achieving strict quality management. Moreover, in the conventional method, the manufacturer of the product is easily tracked and indentified when the product causes any side effects to the user or the aftermath manufacturing management is possible.

Such a conventional method of manufacturing the cosmetics may not satisfy various desires or preferences of the consumers due to the standardization and mass-production thereof. Further, such standardization and mass-production may make it difficult to open the customized cosmetics markets in which the complete product may be tailored to the consumers.

Moreover, the cosmetic company may continuously and unilaterally supply the cosmetics produced not based on preferences or demands or desires of the consumer but based on the needs of the company.

As one example, the primary or base cosmetic components are the same among different complete cosmetics products which have different functional material and/or additives. When the consumer purchase one complete cosmetics product, the consumer should buy a different complete cosmetics product having the different functional material and/or additives although both complete cosmetics products have the same primary or base components and the difference between them results from only small amount of the functional material and/or additives.

For this reason, self-manufacturing of the cosmetics by the consumer using natural materials home is increasing.

However, such self-manufacturing of the cosmetics by the consumer depends on the traditional method without systematic data and is not subject to good quality management. Thus, organic materials without filtering remain in the complete product, to cause harmful germs. Further, when the product is applied to the skin of the consumer for a long time, the germ may invade into the skin cells to lead to skin infections.

As the self-manufacturing of the cosmetics by the consumer using natural materials home gets increasing, operators without certificate increasingly open cosmetic-making classes in do-it-yourself manner. However, source materials used and sold at the cosmetic-making classes may be illegal without authorization by the government, leading to secret sales and social problems.

Therefore, there is a need to develop a cosmetic manufacturing method in which the advantage of the mass-production in a factor is obtained and a legitimate regulation on quality control of the cosmetic product is observed and source materials of the cosmetic product are selected based on usage and/or preferences and/or desires of the consumer, to allow the consumer to be involved in the manufacturing process.

### SUMMARY OF INVENTION

The present disclosure has been conceived with consideration of the foregoing and thus provides a method of manufacturing customized cosmetics in which a base product, functional material and/or additive, as a semi-product, produced or prepared in a factory individually are supplied to a consumer or a cosmetic store wherein the base product has primary components in terms of cosmetic function and thus may be an end-product or complete product, and the consumer selects the functional material and/or additive in accordance with usage, preference and/or skin conditions of the consumer and mix the selected functional material and/or additives with the base product, thereby exhibit the secondary effect of the functional material and/or additive based on the needs of the consumer.

In accordance with one aspect of the present disclosure, there is provided a method of manufacturing a customized cosmetics by using a base product and a functional material and/or additive, the additive including color-rendering agent and/or fragrance-rendering agent, the method comprising: manufacturing, as a semi-product, a base product and functional material and/or additive individually in a factory; selecting, by a consumer, the functional material and/or additive in accordance with usage and/or preference and/or skin conditions of the consumer; and mixing the selected functional material and/or additive into the base product, to produce a complete cosmetics tailored to the consumer, wherein the manufacturing step comprises adjusting moisture percentages of the base product and functional material and additive to be predetermined values respectively so that, after the mixing step, the complete cosmetics has a target moisture percentage, wherein the target moisture is tailored by the consumer or determined based on the skin conditions of the consumer.

In one embodiment, the moisture percentage within each of the base product and functional material and/or additive is determined as follows:

$$a + b + c = d , \\ \frac{a \times 100}{d} + \frac{b \times 100}{d} + \frac{c \times 100}{d} = 100 ( \% )$$

$${x}_{1} = \frac{a \times 100}{d} , \\ {x}_{2} = \frac{b \times 100}{d} , \\ {x}_{3} = \frac{c \times 100}{d}$$

where a, b and c refer to masses of the base product and functional material and additive respectively, d refers to a total mass thereof, and x₁, x₂ and x₃ refer to moisture percentages of the base product and functional material and additive respectively.

In one embodiment, the mixing step further comprises adding a natural extract to a mixture of the selected functional material and/or additive and the base product, the natural extract being selected by the consumer, or mixing the natural extract together the selected functional material and/or additive and the base product.

In one embodiment, the natural extract is acquired using squeezing, crushing, filtering and sterilizing processes.

In one embodiment, the selecting and/or mixing steps are carried out directly by the consumer and/or at a store having as the semi-product, the base product and functional material and/or additive.

In one embodiment, the method may further comprises, before the selecting step, acquiring information about a skin of the consumer via a test or examination of the skin of the consumer, and selecting the functional material and/or additive based on the acquired information.

In accordance with the present disclosure may have following advantages. It should be appreciated that the present disclosure may have not only following advantages but also other advantages and thus a scope of the present disclosure may not limited to the following advantages.

According to the present disclosure, the base product, functional material and/or additive, as a semi-product, produced or prepared in a large quantity in a factory individually are supplied to a consumer or a cosmetic store wherein the base product has primary components in terms of cosmetic function and thus may be an end-product or complete product, and the consumer selects the functional material and/or additive in accordance with usage, preference and/or skin conditions of the consumer and mix the selected functional material and/or additives with the base product, thereby exhibit the secondary effect of the functional material and/or additive based on the needs of the consumer, thereby satisfying various needs of users and enabling each individual to prepare cosmetics tailored to him/her.

### BRIEF DESCRIPTION OF THE DRAWINGS

These and/or other aspects of the invention will become apparent and more readily appreciated from the following description of the embodiments, taken in conjunction with the accompanying drawings of which:

FIG. 1 is a flow chart of a method of manufacturing a customized cosmetics according to the present disclosure;

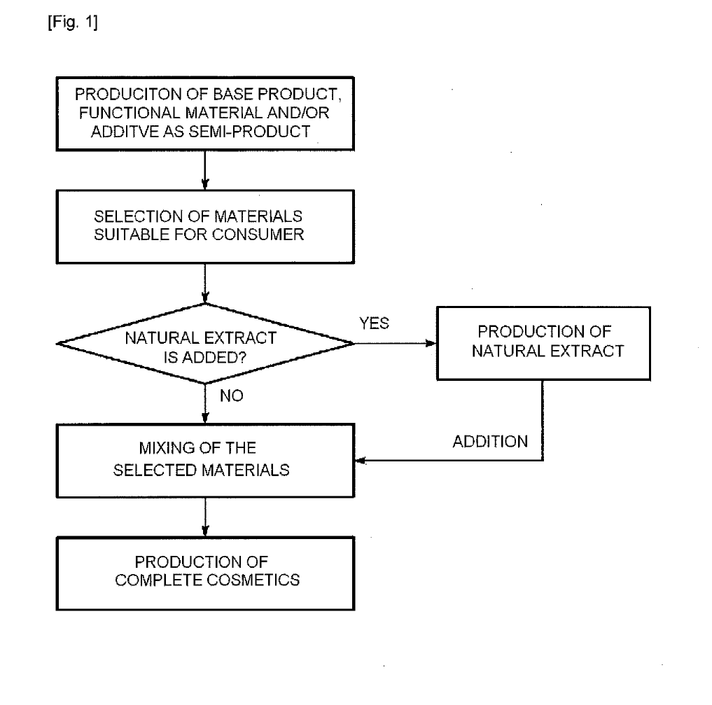

FIG. 2 is a flow chart illustrating in more details the method of FIG. 1;

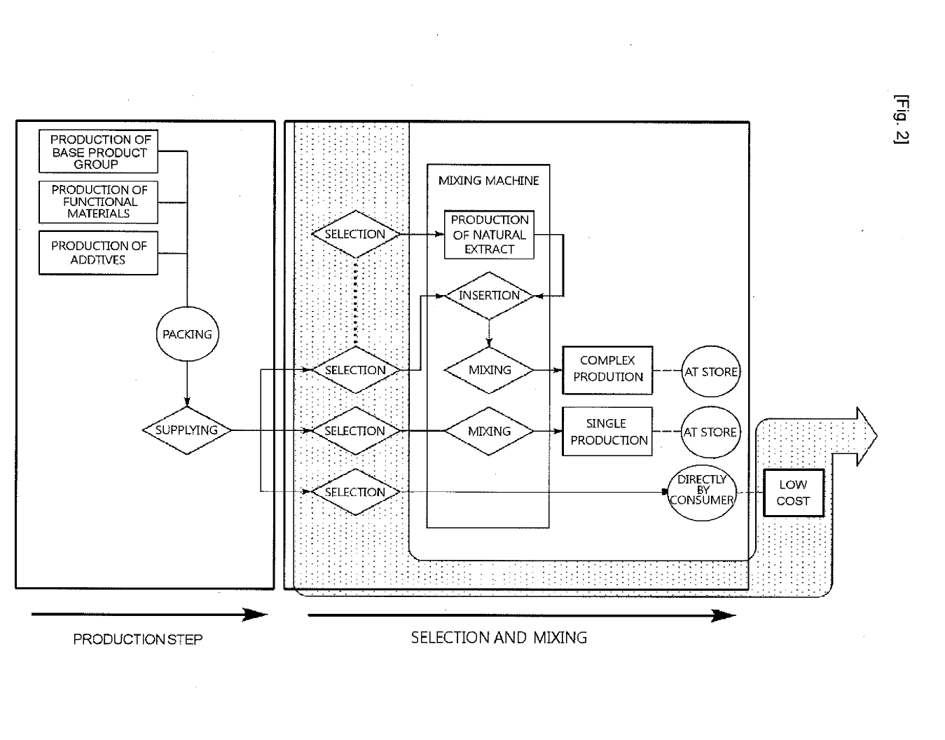

FIG. 3 is a flow chart illustrating detailed processes of manufacturing as a semi-product a base product, functional material and/or additive, according to the present disclosure;

FIG. 4 is a flow chart illustrating process of producing a complete cosmetics at a cosmetic store, according to the present disclosure; and

FIG. 5 illustrates an exemplary table of the base product, functional material and/or additive applied according to the present disclosure.

### DETAILED DESCRIPTIONS OF EMBODIMENTS

The present disclosure may be embodied in many different forms and should not be construed as limited to the embodiments set forth herein. Rather, these embodiments are provided so that this disclosure will be thorough and complete, and will fully convey the scope of the disclosure to those skilled in the art. The present disclosure is defined only by the categories of the claims, and a scope of the present disclosure may include all equivalents to embody a spirit and idea of the present disclosure.

The terminology used in the present disclosure is for the purpose of describing particular embodiments only and is not intended to limit the disclosure. For example, the terminology used in the present disclosure may be construed as follows.

As used in the disclosure and the appended claims, the singular forms “a”, “an” and “the” are intended to include the plural forms as well, unless context clearly indicates otherwise. It will be further understood that the terms “comprise” and/or “comprising and/or “include” and/or “including” and/or “have” and/or “having”” when used in this specification, specify the presence of stated features, integers, steps, operations, elements, and/or components, but do not preclude the presence or addition of one or more other features, integers, steps, operations, elements, components, and/or groups thereof.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

Below, a method of manufacturing a customized cosmetics in accordance with the present disclosure may be in details described with reference to the drawings.

The method of manufacturing customized cosmetics in accordance with the present disclosure is characterized in that a base product, functional material and/or additive, as a semi-product, produced or prepared in a factory individually are supplied to a consumer or a cosmetic store wherein the base product, functional material and/or additive have their unique effects and the consumer selects the functional material and/or additive in accordance with usage, preference and/or skin conditions of the consumer and mix the selected functional material and/or additives with the base product, thereby exhibit the secondary effect of the functional material and/or additive based on the needs of the consumer.

The function material discussed herein may refer to material having specific effects or functions, including, but being limited thereto, materials to exhibit wrinkle removal, anti-cancer, suppression of microbial activity, etc.

The additive discussed herein may include, but be limited thereto, a color-rendering agent to render a specific color from the end-product, and a fragrance-rendering agent to render a specific fragrance from the end-product. The base or primary product discussed herein may refer to a product to decide which type of the end-product mainly is of except for the functional material and additive.

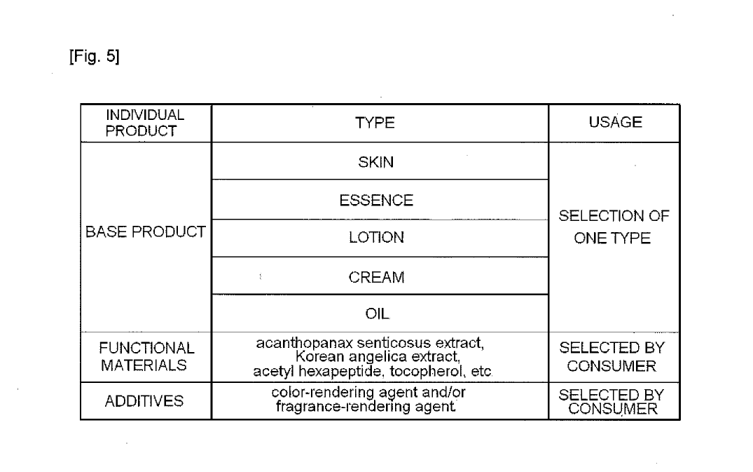

For example, the base or primary product, as shown in FIG. 5, may be, but be limited thereto, of a skin type, essence type, lotion type, cream type, oil type, etc. Further, as an example, the functional material, as shown in FIG. 5 may include, but be limited thereto, acanthopanax senticosus extract, Korean angelica extract, acetyl hexapeptide, tocopherol, etc.

Although the examples of the base product, functional material and additive are shown in FIG. 5, the present disclosure is not limited thereto. Since the definition of the cosmetics has a comprehensive range, the examples of the base product, functional material and additive as shown in FIG. 5 are suggested only as examples to help better understanding of the present disclosure.

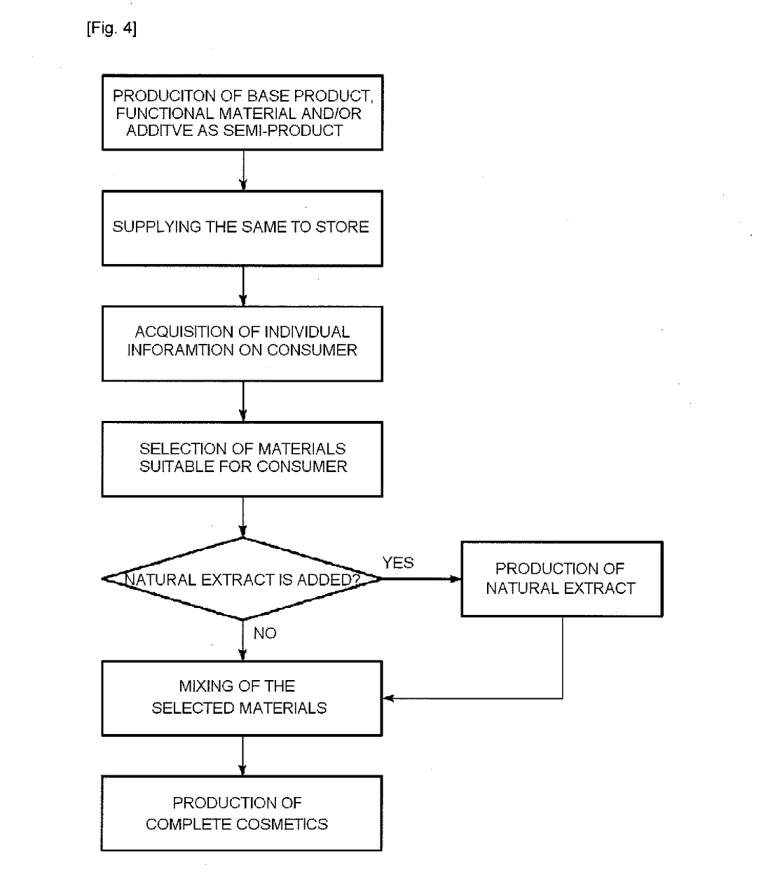

The method of manufacturing customized cosmetics in accordance with the present disclosure may include, as shown in FIG. 4, a first step of manufacturing, as a semi-product, a base product and functional material and/or additive individually in a factory; a second step of selecting, by a consumer, the functional material and/or additive in accordance with usage and/or preference and/or skin conditions of the consumer; and a third step of mixing the selected functional material and/or additive into the base product, to produce a complete cosmetics tailored to the consumer.

At the first step, a base product and functional material and/or additive (hereinafter, referred to as individual product groups) may be manufactured as a semi-product in a large quantity in a factory in the same manner as in a conventional cosmetics manufacturing process.

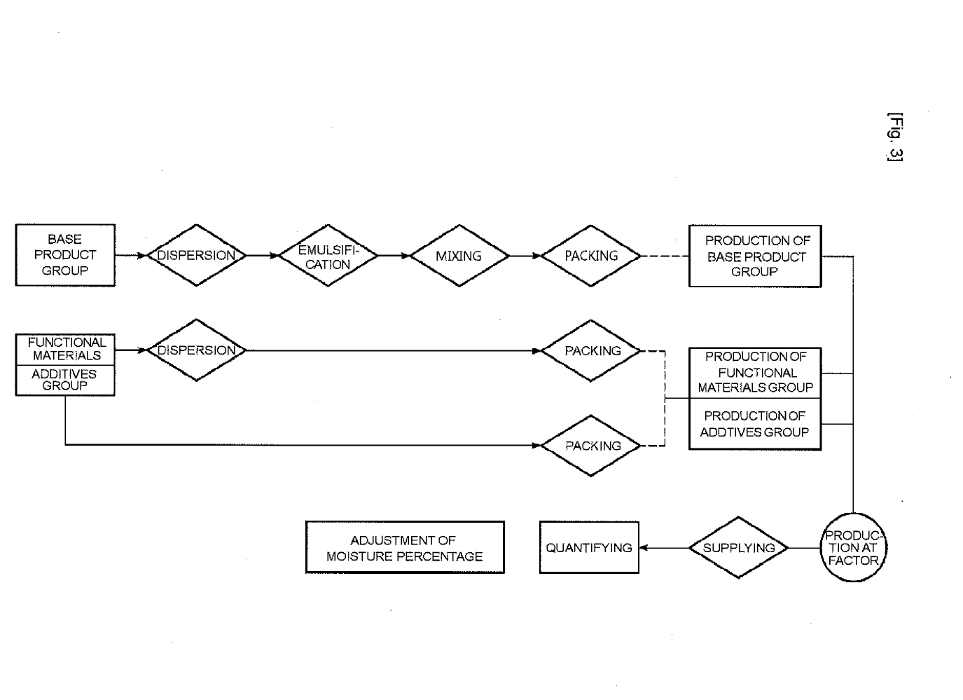

That is, using a series of processes as shown in FIG. 3, the individual product groups may be produced individually with each having a predetermined moisture percentage, the moisture resulting from water and/or oil components within the individual products.

The base product may be produced as a semi-product or a complete product by a process including a dispersion step in which aqueous materials not containing the function material and additive are mixed with purified water at a high speed, an emulsifying step in which the mixture is further mixed with aqueous and oily components at a high temperature and then gets cooled at a room temperature to achieve a stable viscosity, a step in which a preservation agent and/or a pearlescent agent is further mixed into the resulting mixture, a step in which the mixture is adjusted in terms of a moisture percentage and a final step in which the mixture is quantified to be packed as an individual housing.

The produced base product may decide a type of complete cosmetics products, including a skin type, essence type, lotion type, cream type, oil type, etc. As mentioned above, the base product itself may be an end or complete product.

The functional material and/or additive may be subjected to different processes depending on moisture amounts of source materials thereof. For example, when the source materials have small moisture amounts, the moisture amounts thereof may be adjusted to have a predetermined value using a dispersion process.

Otherwise, when the source materials have large moisture amounts, the moisture amounts thereof may be adjusted to have the predetermined value. Thus moisture-adjusted materials are quantified to be packed as an individual housing. At this time, the functional material and/or additive may be produced in accordance preferences and/or needs and/or usage of the consumers.

In this way, in the method of manufacturing customized cosmetics in accordance with the present disclosure, the individual product groups may be produced in a factory in a large quantity as the semi-product, and then may be mixed with each other in accordance with usage and/or preference and/or skin conditions of the consumer.

The first step where the individual product groups may be produced in a factory in a large quantity as the semi-product may include an adjusting step of moisture percentages of the base product and functional material and additive to be predetermined values respectively so that, after the mixing step, the complete cosmetics has a target moisture percentage which may be determined based on the skin conditions of the consumer or may be tailored to the consumer.

That is, at the first step, the moisture amounts of the individual product groups are individually adjusted to have a predetermined value. Hence, when the base product and functional material and/or additive are individually selected by the consumer and then mixed with each other, the resultant complete cosmetics product has the target or appropriate moisture amount. Therefore, this may eliminate a separate process in which the consumer otherwise adjusts the moisture amount of the individual product groups.

In one embodiment, the moisture percentage within each of the base product and functional material and/or additive may be determined as follows:

$$a + b + c = d , \\ \frac{a \times 100}{d} + \frac{b \times 100}{d} + \frac{c \times 100}{d} = 100 ( \% )$$

$${x}_{1} = \frac{a \times 100}{d} , \\ {x}_{2} = \frac{b \times 100}{d} , \\ {x}_{3} = \frac{c \times 100}{d}$$

where a, b and c refer to masses of the base product and functional material and additive respectively, d refers to a total mass thereof, and x₁, x₂ and x₃ refer to moisture percentages of the base product and functional material and additive respectively.

That is, assuming that the moisture percentage of the complete cosmetics product is 100%, each of the moisture percentages of the individual product groups may be such that 100% may be divided by 3 and then be added or subtracted by relative masses of the materials of the individual product groups.

As one example, assuming that the masses of the base product, functional material and additive are 200 g, 100 g, and 50 g respectively, the total mass of the complete cosmetics product being 350 g, each of the moisture percentages of the individual product groups may be adjusted in the first step such that the moisture percentages of the base product, functional material and additive are 57.1%, 28.6% and 14.3% respectively.

These resultant moisture-adjusted products are then selected based on with usage and/or preference and/or skin conditions of the consumer and mixed with each other. In this case, as mentioned above, the resultant complete cosmetics product has the target or appropriate moisture amount because the moisture amounts of the individual product groups have been adjusted to have a predetermined value and are maintained as it is. Therefore, this may eliminate a separate process in which the consumer otherwise adjusts the moisture amount of the individual product groups, allowing the consumer for the easy manufacturing of the tailored cosmetics product. This is true whether the consumer directly manufactures the cosmetics product home or uses a mixing machine of a cosmetics store to make the same.

The consumer may select the individual product groups, namely, the base product and functional material and/or additive based on usage and/or preference and/or skin conditions of the consumer and mix them with each other to obtain the tailored cosmetics product.

More specifically, the consumer may select one of the various base or primary products to define the type of the end product, and may select one or more functional materials in accordance with the desired function or effect and/or the fragrance-rendering agent or color-rendering agent as the additive based on the desire or preference of the consumer and mix the selected base product and functional material and/or additive with each other. In this manner, the consumer may easily make the tailored end-product to him/her in a reliable manner.

At the mixing step, the consumer may employ a variety of mixing machines including, but being limited thereto, an agitator, a mixer, a juice extractor, etc to mix the selected base product and functional material and/or additive with each other. The present applicant discloses an example of such a mixing machine in Korean granted and issued utility model number 20-0412573 filed by the same applicant as in the present application, titled “APPARATUS OF MANUFACTURING NATURAL COSMETICS WITH FILTERING AND STERILIZATION OF NATURAL EXTRACTS”.

Moreover, the he mixing step further comprises adding a natural extract to the mixture of the selected functional material and/or additive and the base product or mixing the natural extract together the selected functional material and/or additive and the base product. The natural extract may be selected by the consumer.

More specifically, the consumer may select natural materials including fruits, vegetables, oriental medicine materials, or the like to add in the end-product. Then, the selected materials may be extracted using squeezing, crushing, filtering and sterilizing processes to acquire a natural extract. The acquired natural extract may be mixed together with the selected functional material and/or additive and the base product. Otherwise, the acquired natural extract may be added into the mixture of the selected functional material and/or additive and the base product.

Examples of the natural extract may be disclosed in Korean granted and issued utility model number 20-0412573 fried by the same applicant as in the present application, titled “APPARATUS OF MANUFACTURING NATURAL COSMETICS WITH FILTERING AND STERILIZATION OF NATURAL EXTRACTS”.

The selecting and/or mixing steps may be carried out directly by the consumer, and/or at a store having as the semi-product, the base product and functional material and/or additive. That is, the consumer may directly select and buy the base product and functional material and/or additive in online or offline manner and directly mix the selected materials for him/her self home. Otherwise, such selection, purchase and/or mixing may be carried out at the cosmetics store.

Further, the method of manufacturing the customized cosmetics may further include, before the selecting step, acquiring information about a skin of the consumer via a test or examination of the skin of the consumer, and selecting the functional material and/or additive based on the acquired information. This is shown in FIG. 4.

The individual semi-products produced or prepared and packaged in a large quantity in a factory individually may be supplied to a cosmetic store or a consumer in an on-line manner.

The consumer may select the base product and functional material and/or additive at the store, namely, in an offline manner or in an online manner using Internet service. In the former case, the consumer may be subject to a test or examination of the skin of the consumer, to acquire information about the skin of the consumer.

The cosmetic specialist at the store may select the base product and functional material and/or additive to be suitable for the consumer based on the acquired information.

In this way, the consumer may be given the base product and functional material and/or additive to be suitable for his/her skin conditions. Then, the selected materials may be mixed with each other to produce the tailored end-products, as mentioned above.

Further, as addressed above, the natural extract selected by the consumer may be mixed together with the selected functional material and/or additive and the base product or, otherwise may be added into the mixture of the selected functional material and/or additive and the base product.

According to the present disclosure, the base product, functional material and/or additive, as a semi-product, produced or prepared in a large quantity in a factory individually are supplied to a consumer or a cosmetic store and the consumer selects the functional material and/or additive in accordance with usage, preference and/or skin conditions of the consumer and mix the selected functional material and/or additives with the base product, thereby exhibit the secondary effect of the functional material and/or additive based on the needs of the consumer, thereby satisfying various needs of users and enabling each individual to prepare cosmetics tailored to him/her.

It will be apparent to those skilled in the art that various modifications and variations can be made in the present invention without departing from the spirit or scope of the inventions. Thus, it is intended that the present invention covers the modifications and variations of this invention provided they come within the scope of the appended claims and their equivalents.

What is claimed is:

1. A method of manufacturing a customized cosmetics by using a base product and a functional material and/or additive, the additive including color-rendering agent and/or fragrance-rendering agent, the method comprising:
manufacturing, as a semi-product, a base product and functional material and/or additive individually in a factory;
selecting, directly by a consumer or at a cosmetic store, the functional material and/or additive in accordance with usage and/or preference and/or skin conditions of the consumer; and
mixing the selected functional material and/or additive into the base product, to produce a complete cosmetics tailored to the consumer,
wherein the manufacturing step comprises adjusting moisture percentages of the base product and functional material and additive to be predetermined values respectively so that, after the mixing step, the complete cosmetics has a target moisture percentage.

2. The method of claim 1, wherein the moisture percentage within each of the base product and functional material and/or additive is determined as follows:
$$a + b + c = d , \\ \frac{a \times 100}{d} + \frac{b \times 100}{d} + \frac{c \times 100}{d} = 100 ( \% )$$
$${x}_{1} = \frac{a \times 100}{d} , \\ {x}_{2} = \frac{b \times 100}{d} , \\ {x}_{3} = \frac{c \times 100}{d}$$
where a, b and c refer to masses of the base product and functional material and additive respectively, d refers to a total mass thereof, and x₁, x₂ and x₃ refer to moisture percentages of the base product and functional material and additive respectively.

3. The method of claim 1, wherein the mixing step further comprises adding a natural extract to a mixture of the selected functional material and/or additive and the base product or mixing the natural extract together with the selected functional material and/or additive and the base product, wherein the natural extract is selected by the consumer.

4. The method of claim 3, wherein the natural extract is acquired using squeezing, crushing, filtering and sterilizing processes.

5. The method of claim 1, wherein the selecting and/or mixing step are carried out directly by the consumer and/or at a store having as the semi-product, the base product and functional material and/or additive.

6. The method of claim 5, further comprising, before the selecting step, acquiring information about a skin of the consumer via a test or examination of the skin of the consumer, and selecting the functional material and/or additive based on the acquired information.
